(19) United States
(12) Patent Application Publication
(10) Pub. No.: US 2011/0311641 A1
(43) Pub. Date: Dec. 22, 2011

(54) COMPOSITION FOR MAKING PATHOLOGICAL TISSUES DEHYDRATION AND/OR CONTRACTION

(76) Inventors: Kuok Leong Tam, Macao (CN); Io cheng Tam, Macao (CN); Hio man Tam, Macao (CN)

(21) Appl. No.: 13/060,141

(22) PCT Filed: Aug. 24, 2009
(86) PCT No.: PCT/CN09/73442

§ 371 Date: Sep. 8, 2011

(30) Foreign Application Priority Data
Aug. 22, 2008 (CN) 200810030331.4

(51) Int. Cl. A61K 33/26; C01G 49/14; A61P 29/00; A61P 23/02; A61K 35/56; A61P 17/02

(52) U.S. Cl. 424/537; 424/647; 423/558

(57) ABSTRACT

The present invention provides a pharmaceutical composition for dehydrating and atrophying pathological tissues comprising ferric subsulfate, polyferric sulfate or a mixture thereof is disclosed. Ferric subsulfate and polyferric sulfate are traditionally used as water treatment agent. The inventor surprisingly discovered the medical value of these substances. The compositions are cost-effective, convenient to use and have significant effects in treatment of vascular diseases such as hemorrhoids, hemangiomas, hematomas (including hematomas caused by brain hemorrhage), thrombi and varices, and topical traumas such as bleeding and edemas. The compositions are also capable of killing various tumor cells by targeted injection.

### CROSS-REFERENCE TO RELATED APPLICATION

The present application claims priority from Chinese patent application number 200810030331.4, entitled “PHARMACEUTICAL COMPOSITIONS FOR DEHYDRATING AND ATROPHYING PATHOLOGICAL TISSUES” and filed in the name of Kuok Leong TAM on Aug. 22, 2008. The entire content of the application is incorporated herein by reference.

### FIELD OF THE INVENTION

The present invention relates to pharmaceutical compositions for dehydrating and atrophying pathological tissues.

### BACKGROUND OF THE INVENTION

Ferric subsulfate, a compound having a molecular formula Fe₄(OH)₂(SO₄)₅, is a pale-yellow particle when it is in solid form. The particle is easily soluble in water to form a reddish-brown solution which can be crystallized and solidified at low temperature. The compound is obtained by reacting ferrous sulfate with heated dilute sulfuric acid and dilute nitric acid, and normally used as mordant or waste water treatment agent.

Polyferric sulfate (PFS) is a polymer having a molecular formula [Fe₂(OH)ₙ(SO₄)_{3-n/2}]m. The polymer has been used as inorganic flocculating agent and has several advantages compared with other various flocculating agents, for example, relatively low dosage, adapt to broad pH range, high impurity (turbidity, COD, suspended matters, etc.) removal rate, low concentration of residues, high floc settling velocity, good decolorization effect, etc. The polymer is widely used for purification of industrial waste water, urban sewage, industrial water and domestic water.

Ferric subsulfate, polyferric sulfate or a mixture thereof has never been found worldwide in medical field to dehydrate and atrophy pathological tissue.

### SUMMARY OF THE INVENTION

An object of the present invention is to provide a pharmaceutical composition for dehydrating and atrophying pathological tissues, comprising ferric subsulfate or polyferric sulfate as active component.

In order to achieve the above object, a pharmaceutical composition for dehydrating and atrophying pathological tissues comprising ferric subsulfate, polyferric sulfate or a mixture thereof is provided.

Polyferric sulfate is a polymer of ferric subsulfate. Ferric subsulfate, polyferric sulfate or a mixture in any ratio thereof is capable of enabling pathological tissues to become dehydrated and necrotic, which are subsequently absorbed by, or form a scab and slough from normal tissues. The composition is effective in treatment of vascular diseases such as hemorrhoids, hemangiomas, hematomas, thrombi and varices, topical traumas such as bleeding and edemas, and tumors.

Preferably, the composition is an aqueous solution comprising as active component ferric subsulfate, polyferric sulfate or a mixture thereof in an amount of at least 2.5% by weight. Of course, the concentration of the solution cannot be too high because the solution becomes saturated when the weight percent of the active component reaches about 45%.

Preferably, the solution further comprises an anesthetic for alleviating pains.

Preferably, the anesthetic is lidocaine hydrochloride, mint, toad venom, procaine or dicaine.

Preferably, the solution contains 2.5˜15 wt. % of polyferric sulfate, 0.25 wt. % of mint, and balanced amount of water. This solution is suitable for treating topical traumas such as traumatic wounds, surgical wounds, inflammatory wounds, suppurative wounds and injuries caused by chemical agents or cytotoxins.

Preferably, the aqueous solution contains 2.5˜15 wt. % of polyferric sulfate, 2 wt. % of lidocaine hydrochloride, and balanced amount of water. The solution can be prepared into injection for treating various hemorrhoids.

Preferably, the aqueous solution contains saturated amount of ferric subsulfate, 0.5 wt. % of toad venom, and balanced amount of water. This solution is suitable for topical application, particularly for treating capillary hemangiomas and nevus vascularis, such as cherry angiomas on skin or scrotum (also known as senile angioma or senile nevus vascularis).

Preferably, the composition is a solid powder comprising as active component ferric subsulfate, polyferric sulfate or a mixture thereof in an amount of 10˜60% by weight, and balanced amount of solid excipient. Excipients are used to dilute the composition and can be selected from silicon dioxide and medical starch, etc. The powder can be applied on bleeding traumatic wounds or suppurative wounds, so that the wounds will form a scab and be cured in a short time.

Preferably, the composition is a paste comprising as active component nanoparticles of ferric subsulfate, polyferric sulfate or a mixture thereof in an amount of 5˜15 wt. %, and balanced amount of excipient. Excipients can be Vaseline oil, etc.

The present invention is a use invention. Ferric subsulfate and polyferric sulfate are traditionally used as water treatment agent. The present inventor surprisingly discovered the medical value of these substances. A large amount of experiments were performed over years and demonstrated very good effect of the substances. These compositions are cost-effective, convenient to use and have significant effects in treatment of vascular diseases such as hemorrhoids, hemangiomas, hematomas (including hematomas caused by brain hemorrhage), thrombi and varices, and topical traumas such as bleeding and edemas. The compositions are also capable of killing various tumor cells by targeted injection.

### DETAILED DESCRIPTION OF THE INVENTION

### EXAMPLE 1

A small amount of solid powder of ferric subsulfate is applied onto a bleeding traumatic wound. The wound quickly forms a scab within 5 seconds, but the patient feels a bit painful.

### EXAMPLE 2

A small amount of solid powder of polyferric sulfate is applied onto a bleeding traumatic wound. The wound quickly forms a scab within 5 seconds, but the patient feels a bit painful.

It is proved that both ferric subsulfate and polyferric sulfate can cause traumatic wound to quickly form a scab. These two substances do not react with each other, and thus it is expected that their mixture has the same function.

### EXAMPLE 3

60 grams of solid powder of ferric subsulfate are well mixed with 40 grams of medical starch. A small amount of the mixture is applied onto a bleeding traumatic wound. The wound is further bound up by gauze. The wound forms a scab after 10 minutes, and the patient feels less painful.

### EXAMPLE 4

35 grams of solid powder of ferric subsulfate are well mixed with 65 grams of medical silicon dioxide. A small amount of the mixture is applied onto a bleeding traumatic wound. The wound is further bound up by gauze. The wound forms a scab after half an hour, and the patient feels less painful.

### EXAMPLE 5

10 grams of solid powder of ferric subsulfate are well mixed with 90 grams of medical silicon dioxide. A small amount of the mixture is applied onto a bleeding traumatic wound. The wound is further bound up by gauze. The wound forms a scab after two hours, and the patient feels much less painful.

### EXAMPLE 6

15 grams of ferric subsulfate are processed into nanoparticles and well mixed with 85 grams of Vaseline oil to form a paste mixture. A small amount of the mixture is applied onto a bleeding traumatic wound. The wound is further bound up by gauze. The wound forms a scab after 10 minutes, and the patient feels less painful.

### EXAMPLE 7

10 grams of ferric subsulfate are processed into nanoparticles and well mixed with 90 grams of Vaseline oil to form a paste mixture. A small amount of the mixture is applied onto a bleeding traumatic wound. The wound is further bound up by gauze. The wound forms a scab after half an hour, and the patient feels less painful.

### EXAMPLE 8

5 grams of ferric subsulfate are processed into nanoparticles and well mixed with 95 grams of Vaseline oil to form a paste mixture. A small amount of the mixture is applied onto a bleeding traumatic wound. The wound is further bound up by gauze. The wound forms a scab after half an hour, and the patient feels much less painful.

It should be noted that, both ferric subsulfate and polyferric sulfate are very hygroscopic. When exposed in air, they will agglomerate and lose efficacy within minutes. Therefore, powders, pastes and creams must be stored in a sealed condition.

Powders can be packed in ampoules or in a small bag for single use. Pastes and creams are fit to be packed in small canisters, preferably also for single use.

### EXAMPLE 9

2.5 grams of solid powder of ferric subsulfate are dissolved in 97.5 grams of distilled water and filtered by a bacteria filter to obtain ferric subsulfate solution I. The solution I is then injected into a hematoma with an amount of ⅕ of the volume of the pathological tissues. The hematoma reduces in size within 5 minutes.

### EXAMPLE 10

20 grams of solid powder of ferric subsulfate are dissolved in 80 grams of distilled water and filtered by a bacteria filter to obtain ferric subsulfate solution II. The solution II is then used to treat traumatic wounds, surgical wounds, inflammatory wounds, suppurative wounds and local injuries caused by chemical agents or cytotoxins by killing bacteria and viruses.

### EXAMPLE 11

Ferric subsulfate is dissolved in distilled water to obtain a saturated subsulfate solution. The solution is then used to treat traumatic wounds, surgical wounds, inflammatory wounds, suppurative wounds and injuries caused by chemical agents or cytotoxins by killing bacteria and viruses and promoting curing.

Examples 3 to 12 are repeated except that ferric subsulfate is replaced with polyferric sulfate or a mixture of ferric subsulfate and polyferric sulfate. Substantially same results are obtained.

### EXAMPLE 12

2.5 grams of polyferric sulfate, 0.25 grams of mint and 97.25 grams of distilled water are well mixed. The resulting solution can be used for sterilization and treatment of traumas, and the patient feels very little pain.

### EXAMPLE 13

10 grams of polyferric sulfate, 0.25 grams of mint and 89.25 grams of distilled water are well mixed. The resulting solution is used for treatment of traumas, and the patient feels very little pain. The solution shows a significant sterilization effect.

### EXAMPLE 14

15 grams of polyferric sulfate, 0.25 grams of mint and 84.25 grams of distilled water are well mixed. The resulting solution is used for treatment of traumas, and the patient feels very little pain. The solution shows a significant sterilization effect.

### EXAMPLE 15

10 grams of polyferric sulfate, 0.25 grams of lidocaine hydrochloride and 88 grams of distilled water are well mixed. The resulting solution is used for treatment of traumas, and the patient feels very little pain. The solution shows a significant sterilization effect.

The above compositions can be injected into various hemorrhoids such as internal hemorrhoids, external hemorrhoids, mixed hemorrhoids, thrombosed hemorrhoids, infected hemorrhoids and rectal prolapse hemorrhoids, hemangiomas, hematomas, thrombi and varices with an amount of one fifth of the volume of the hemorrhoids, hemangiomas, hematomas, thrombi or varices.

The injection procedure comprises steps of cleaning and sterilizing the affected parts; injecting the solution into the hemorrhoids, hemangiomas, hematomas or thrombi at several points; pressing the affected parts with cotton swab to evenly distribute the solution, the hemorrhoids, hemangiomas or thrombi becoming stiff within seconds. The hemangiomas and hematomas began to soften, dehydrate, atrophy and disappear after three minutes or more. The dehydration and atrophying process completed within ten to twenty minutes. After seven to fourteen days, the pathological tissues were either substituted by normal tissues or sloughed, and the wounds were cured. No co-effect, bleeding, scar or any other side effect was observed. Therefore, this method is a novel dehydration therapy for hemorrhoids, hemangiomas, hematomas, and thrombi. The pharmaceutical compositions have very specific, rapid and excellent effect in dehydration and atrophying of the blood stasis pathological tissues caused by the varix in hemorrhoids; hematomas; and thrombi, which was not found in literatures worldwide.

### EXAMPLE 16

Example 15 is repeated except that the amount of polyferric sulfate is changed to 2.5 wt. %. Same results were observed.

### EXAMPLE 17

Example 15 is repeated except that the amount of polyferric sulfate is changed to 15 wt. %. More rapid effects were observed.

### EXAMPLE 18

Ferric subsulfate is mixed with distilled water to form a ferric subsulfate saturated solution with the ferric subsulfate in an amount of approximately 45 wt. %. 0.5 wt. % of toad venom is added to the solution, stirred and dissolved. The resulting mixture is then subpackaged and reserved.

The above pharmaceutical composition is particularly suitable for treating surface capillary hemangiomas and nevus vascularis, such as cherry angiomas on skin or scrotum (also known as senile angioma or senile nevus vascularis). Tens to hundreds of surface capillary hemangiomas and nevus vascularis can be treated at one time. The treatment procedures may be carried out as follows. The pharmaceutical composition is first applied on all hemangiomas and nevi to be treated. After tens of seconds, the surfaces of the hemangiomas and nevi are anaesthetized. A sterilized thick steel needle is used to puncture the hemangiomas and nevi to make a dozen of holes. The pharmaceutical composition is then injected into the hemangiomas and nevi. Blood in the hemangiomas and nevi coagulates at once, and the hemangiomas and nevi begin to soften, dehydrate and atrophy. It only takes ten to twenty seconds to treat one hemangioma or nevus, so that more than 167 small hemangiomas can be treated by two doctors in an hour. Seven to ten days later, the pathological tissues form a scab and slough, and the tissues are cured without leaving any scars.

What is claimed is:

1. A pharmaceutical composition for dehydrating and atrophying pathological tissues, comprising ferric subsulfate, polyferric sulfate or a mixture thereof.

2. The pharmaceutical composition for dehydrating and atrophying pathological tissues of claim 1, wherein the composition is an aqueous solution comprising ferric subsulfate, polyferric sulfate or a mixture thereof in an amount of at least 2.5% by weight.

3. The pharmaceutical composition for dehydrating and atrophying pathological tissues of claim 2, wherein the aqueous solution further comprises an anesthetic.

4. The pharmaceutical composition for dehydrating and atrophying pathological tissues of claim 3, wherein the anesthetic is lidocaine hydrochloride, mint, toad venom, procaine or dicaine.

5. The pharmaceutical composition for dehydrating and atrophying pathological tissues of claim 4, wherein the aqueous solution contains 2.5˜15 wt. % of polyferric sulfate, 0.25 wt. % of mint, and balanced amount of water.

6. The pharmaceutical composition for dehydrating and atrophying pathological tissues of claim 4, wherein the aqueous solution contains 2.5˜15 wt. % of polyferric sulfate, 2 wt. % of lidocaine hydrochloride, and balanced amount of water.

7. The pharmaceutical composition for dehydrating and atrophying pathological tissues of claim 4, wherein the aqueous solution contains saturated amount of ferric subsulfate, 0.5 wt. % of toad venom, and balanced amount of water.

8. The pharmaceutical composition for dehydrating and atrophying pathological tissues of claim 1, wherein the composition is a solid powder comprising as active component ferric subsulfate, polyferric sulfate or a mixture thereof in an amount of 10˜60% by weight, and balance amount of solid excipients.

9. The pharmaceutical composition for dehydrating and atrophying pathological tissues of claim 1, wherein the composition is a paste comprising as active component nanoparticles of ferric subsulfate, polyferric sulfate or a mixture thereof in an amount of 5˜15 wt. %, and balanced amount of excipient.

10. A usage of ferric subsulfate and/or polyferric sulfate on the production of a pharmaceutical composition for dehydrating and atrophying pathological tissues.

11. The usage of claim 10, wherein said pharmaceutical composition is used for treatment of vascular diseases.

12. The usage of claim 10, wherein said pharmaceutical composition is used for treatment of hemorrhoids, hemangiomas, hematomas, thrombi or varices.

13. The usage of claim 10, wherein said pharmaceutical composition is used for treatment of capillary hemangiomas and nevus vascularis.

14. The usage of claim 10, wherein said pharmaceutical composition is used for treatment of cherry angiomas on skin or scrotum.

15. The usage of claim 10, wherein said pharmaceutical composition is used for treatment of various traumatic wounds, surgical wounds, bleeding traumatic wound, inflammatory wounds, or suppurative wounds.

16. The usage of claim 10, wherein said pharmaceutical composition is used for treatment of local injuries caused by chemical agents or cytotoxins.
